SEQUENCE LISTING (15 sequences)

SEQ ID NO: 1 - protein, 6 aa, Artificial sequence
  Synthetic construct
LPFNQL

SEQ ID NO: 2 - protein, 6 aa, Artificial sequence
  Synthetic construct
LPFSQL

SEQ ID NO: 3 - protein, 17 aa, Artificial sequence
  Synthetic construct
YQEPVLGPVRGPFPIIV

SEQ ID NO: 4 - protein, 14 aa, Artificial sequence
  Synthetic construct
PVLGPVRGPFPIIV

SEQ ID NO: 5 - protein, 8 aa, Artificial sequence
  Synthetic construct
RGPFPIIV

SEQ ID NO: 6 - DNA, 1795 bp, Homo sapiens
ggttaccacttggaaaacaattcatctgaaagaagcacagattttctcatctatcctgtcaacaaagaaagaatcaagagagcaaggacagtgatttcccccgcattgcatttgtcttgaagatcagtcagaaagagaaactcggcatcatctgtgacagacagtggaacgaaaaatgccagtgaaatgctgtttctacaggtcaccaactgcggaaacaatgacatggtctgaaaatatggacacgcttttagccaaccaagctggtctagatgcttttcgaatatttctaaaatcagagtttagtgaagaaaatgttgagttctggcttgcctgtgaagactttaagaaaacgaaaaatgcagacaaaattgcttccaaagccaagatgatttattctgaattcattgaagctgatgcacctaaagagattaacattgacttcggtaccagagacctcatctcaaagaatattgctgaaccaacactcaaatgctttgatgaggctcagaaattaatctattgtctcatggccaaggattctttccctcgatttctgaagtcagagatttataaaaaactggtaaatagccaacaggttccaaatcataaaaaatggctcccttttttgtgaggaaggtaaaagttaactaatcactatacttcagggctacaatattttaaatatacaagcatgatgcattgtcttttgttttgtttttaggatttagaaaacattttttacccaaacagatgaataacgttttatacaacaagcctgaatttctaactcagttgtttagaatgtatttgctttaccagctatttaatctcctactgggggagtacaaagaaagtttatagagatacaatatagtcttaaaccaaaactgaatattcttattatattataatgtaaggaattatacacatcttcacgtggcagaatgaaagacttttgagcatcatatacacaattttaaataccattgctttattcaaaaaaatctcacttttgtaaaaagagaatttctgaaccaaaatacaagttttcatttaatatatttaactgttttttttctgccatttctttccaactatttctaataatgtggttatgaaaactgctacgcctctcaaattatattttttaaatcacaggaatgtatacacatttatatgtatgtcttgaatgcaccatggaccaaagtttttcaaaatatatcacttggctcaattcaatggcatcacatataaaatgtgatgagttatgtatgaaaaggcctcaagggtggggaatactgattttcttatgttaacagaaatataaaagaaagtggaagactaaggagcatagataaatccttataagatgaagtatatagcaagtcataaaatttaagaatttgcaacattatctactcaattgtggggaagtatctattcactccttcagcactgatacttgtttataaaacccaaacaatttttaaatgcatttattttgagatgttcctaaaattgtttcattctatatgtaaatatcctgtgataaatacgaataatttcatttcaatatgagaagctgtaaagattcaacagatctcccacgtttccattttctttgcacagatttatttatctgcattgatatttctgcttttagattgtttgaacattaaaaaatggaggaaaaatagcatggcttattttatgttttcacaaactactcatttgatagacaaaattttgtcttcccttcatcatgagaaataaacatttaaacatattcaaa

SEQ ID NO: 7 - protein, 152 aa, Homo sapiens
MPVKCCFYRSPTAETMTWSENMDTLLANQAGLDAFRIFLKSEFSEENVEFWLACEDFKKTKNADKIASKAKMIYSEFIEADAPKEINIDFGTRDLISKNIAEPTLKCFDEAQKLIYCLMAKDSFPRFLKSEIYKKLVNSQQVPNHKKWLPFL

SEQ ID NO: 8 - DNA, 930 bp, Homo sapiens
atgataacttttctgcccatcattttttccattctaatagtggttacatttgtgattggaaattttgctaatggcttcatagcattggtaaattccattgagtggttcaagagacaaaagatctcttttgctgaccaaattctcactgctctggcagtctccagagttggtttactctgggtattagtattaaattggtatgcaactgagttgaatccagcttttaacagtatagaagtaagaattactgcttacaatgtctgggcagtaatcaaccatttcagcaactggcttgctactagcctcagcatattttatttgctcaagattgccaatttctccaaccttatttttcttcacttaaagaggagagttaagagtgttgttctggtgatactattggggcctttgctatttttggtttgtcatctttttgtgataaacatgaatcagattatatggacaaaagaatatgaaggaaacatgacttggaagatcaaactgaggagtgcaatgtacctttcaaatacaacggtaaccatcctagcaaacttagttcccttcactctgaccctgatatcttttctgctgttaatctgttctctgtgtaaacatctcaaaaagatgcagctccatggcaaaggatctcaagatcccagcatgaaggtccacataaaagctttgcaaactgtgacctccttcctcttgttatgtgccatttactttctgtccataatcatgtcagtttggagttttgagagtctggaaaacaaacctgtcttcatgttctgcgaagctattgcattcagctatccttcaacccacccattcatcctgatttggggaaacaagaagctaaagcagacttttctttcagttttgtggcatgtgaggtactgggtgaaaggagagaagccttcatcttcatag

SEQ ID NO: 9 - protein, 309 aa, Homo sapiens
MITFLPIIFSILIVVTFVIGNFANGFIALVNSIEWFKRQKISFADQILTALAVSRVGLLWVLVLNWYATELNPAFNSIEVRITAYNVWAVINHFSNWLATSLSIFYLLKIANFSNLIFLHLKRRVKSVVLVILLGPLLFLVCHLFVINMNQIIWTKEYEGNMTWKIKLRSAMYLSNTTVTILANLVPFTLTLISFLLLICSLCKHLKKMQLHGKGSQDPSMKVHIKALQTVTSFLLLCAIYFLSIIMSVWSFESLENKPVFMFCEAIAFSYPSTHPFILIWGNKKLKQTFLSVLWHVRYWVKGEKPSSS

SEQ ID NO: 10 - DNA, 1143 bp, Homo sapiens
cctttctgcactgggtggcaaccaggtctttagattagccaactagagaagagaagtagaatagccaattagagaagtgacatcatgttgactctaactcgcatccgcactgtgtcctatgaagtcaggagtacatttctgttcatttcagtcctggagtttgcagtggggtttctgaccaatgccttcgttttcttggtgaatttttgggatgtagtgaagaggcaggcactgagcaacagtgattgtgtgctgctgtgtctcagcatcagccggcttttcctgcatggactgctgttcctgagtgctatccagcttacccacttccagaagttgagtgaaccactgaaccacagctaccaagccatcatcatgctatggatgattgcaaaccaagccaacctctggcttgctgcctgcctcagcctgctttactgctccaagctcatccgtttctctcacaccttcctgatctgcttggcaagctgggtctccaggaagatctcccagatgctcctgggtattattctttgctcctgcatctgcactgtcctctgtgtttggtgcttttttagcagacctcacttcacagtcacaactgtgctattcatgaataacaatacaaggctcaactggcagattaaagatctcaatttattttattcctttctcttctgctatctgtggtctgtgcctcctttcctattgtttctggtttcttctgggatgctgactgtctccctgggaaggcacatgaggacaatgaaggtctataccagaaactctcgtgaccccagcctggaggcccacattaaagccctcaagtctcttgtctcctttttctgcttctttgtgatatcatcctgtgttgccttcatctctgtgcccctactgattctgtggcgcgacaaaataggggtgatggtttgtgttgggataatggcagcttgtccctctgggcatgcagccatcctgatctcaggcaatgccaagttgaggagagctgtgatgaccattctgctctgggctcagagcagcctgaaggtaagagccgaccacaaggcagattcccggacactgtgctgagaatggacatgaaatgagctcttcattaatacgcctgtgagtcttcataaatatgcc

SEQ ID NO: 11 - protein, 333 aa, Homo sapiens
MLTLTRIRTVSYEVRSTFLFISVLEFAVGFLTNAFVFLVNFWDVVKRQALSNSDCVLLCLSISRLFLHGLLFLSAIQLTHFQKLSEPLNHSYQAIIMLWMIANQANLWLAACLSLLYCSKLIRFSHTFLICLASWVSRKISQMLLGIILCSCICTVLCVWCFFSRPHFTVTTVLFMNNNTRLNWQIKDLNLFYSFLFCYLWSVPPFLLFLVSSGMLTVSLGRHMRTMKVYTRNSRDPSLEAHIKALKSLVSFFCFFVISSCVAFISVPLLILWRDKIGVMVCVGIMAACPSGHAAILISGNAKLRRAVMTILLWAQSSLKVRADHKADSRTLC

SEQ ID NO: 12 - DNA, 2521 bp, Homo sapiens
catggggcccagggcaaagaccatctcctccctgttcttcctcctatgggtcctggctgagccggctgagaactcggacttctacctgcctggggattacctcctgggtggcctcttctccctccatgccaacatgaagggcattgttcaccttaacttcctgcaggtgcccatgtgcaaggagtatgaagtgaaggtgataggctacaacctcatgcaggccatgcgctttgcggtggaggagatcaacaatgacagcagcctgctgcctggtgtgctgctgggctatgagatcgtggatgtgtgctacatctccaacaatgtccagccggtgctctacttcctggcacacgaggacaacctccttcccatccaagaggactacagtaactacatttcccgtgtggtggctgtcattggccctgacaactccgagtctgtcatgactgtggccaacttcctctccctatttctccttccacagatcacctacagcgccatcagcgatgagctgcgagacaaggtgcgcttcccggctttgctgcgtaccacacccagcgccgaccaccacatcgaggccatggtgcagctgatgctgcacttccgctggaactggatcattgtgctggtgagcagcgacacctatggccgcgacaatggccagctgcttggcgagcgcgtggcccggcgcgacatctgcatcgccttccaggagacgctgcccacactgcagcccaaccagaacatgacgtcagaggagcgccagcgcctggtgaccattgtggacaagctgcagcagagcacagcgcgcgtcgtggtcgtgttctcgcccgacctgaccctgtaccacttcttcaatgaggtgctgcgccagaacttcactggcgccgtgtggatcgcctccgagtcctgggccatcgacccggtcctgcacaacctcacggagctgcgccacttgggcaccttcctgggcatcaccatccagagcgtgcccatcccgggcttcagtgagttccgcgagtggggcccacaggctgggccgccacccctcagcaggaccagccagagctatacctgcaaccaggagtgcgacaactgcctgaacgccaccttgtccttcaacaccattctcaggctctctggggagcgtgtcgtctacagcgtgtactctgcggtctatgctgtggcccatgccctgcacagcctcctcggctgtgacaaaagcacctgcaccaagagggtggtctacccctggcagctgcttgaggagatctggaaggtcaacttcactctcctggaccaccaaatcttcttcgacccgcaaggggacgtggctctgcacttggagattgtccagtggcaatgggaccggagccagaatcccttccagagcgtcgcctcctactaccccctgcagcgacagctgaagaacatccaagacatctcctggcacaccatcaacaacacgatccctatgtccatgtgttccaagaggtgccagtcagggcaaaagaagaagcctgtgggcatccacgtctgctgcttcgagtgcatcgactgccttcccggcaccttcctcaaccacactgaagatgaatatgaatgccaggcctgcccgaataacgagtggtcctaccagagtgagacctcctgcttcaagcggcagctggtcttcctggaatggcatgaggcacccaccatcgctgtggccctgctggccgccctgggcttcctcagcaccctggccatcctggtgatattctggaggcacttccagacacccatagttcgctcggctgggggccccatgtgcttcctgatgctgacactgctgctggtggcatacatggtggtcccggtgtacgtggggccgcccaaggtctccacctgcctctgccgccaggccctctttcccctctgcttcacaatctgcatctcctgtatcgccgtgcgttctttccagatcgtctgcgccttcaagatggccagccgcttcccacgcgcctacagctactgggtccgctaccaggggccctacgtctctatggcatttatcacggtactcaaaatggtcattgtggtaattggcatgctggccacgggcctcagtcccaccacccgtactgaccccgatgaccccaagatcacaattgtctcctgtaaccccaactaccgcaacagcctgctgttcaacaccagcctggacctgctgctctcagtggtgggtttcagcttcgcctacatgggcaaagagctgcccaccaactacaacgaggccaagttcatcaccctcagcatgaccttctatttcacctcatccgtctccctctgcaccttcatgtctgcctacagcggggtgctggtcaccatcgtggacctcttggtcactgtgctcaacctcctggccatcagcctgggctacttcggccccaagtgctacatgatcctcttctacccggagcgcaacacgcccgcctacttcaacagcatgatccagggctacaccatgaggagggactag

SEQ ID NO: 13 - protein, 839 aa, Homo sapiens
MGPRAKTISSLFFLLWVLAEPAENSDFYLPGDYLLGGLFSLHANMKGIVHLNFLQVPMCKEYEVKVIGYNLMQAMRFAVEEINNDSSLLPGVLLGYEIVDVCYISNNVQPVLYFLAHEDNLLPIQEDYSNYISRVVAVIGPDNSESVMTVANFLSLFLLPQITYSAISDELRDKVRFPALLRTTPSADHHIEAMVQLMLHFRWNWIIVLVSSDTYGRDNGQLLGERVARRDICIAFQETLPTLQPNQNMTSEERQRLVTIVDKLQQSTARVVVVFSPDLTLYHFFNEVLRQNFTGAVWIASESWAIDPVLHNLTELRHLGTFLGITIQSVPIPGFSEFREWGPQAGPPPLSRTSQSYTCNQECDNCLNATLSFNTILRLSGERVVYSVYSAVYAVAHALHSLLGCDKSTCTKRVVYPWQLLEEIWKVNFTLLDHQIFFDPQGDVALHLEIVQWQWDRSQNPFQSVASYYPLQRQLKNIQDISWHTINNTIPMSMCSKRCQSGQKKKPVGIHVCCFECIDCLPGTFLNHTEDEYECQACPNNEWSYQSETSCFKRQLVFLEWHEAPTIAVALLAALGFLSTLAILVIFWRHFQTPIVRSAGGPMCFLMLTLLLVAYMVVPVYVGPPKVSTCLCRQALFPLCFTICISCIAVRSFQIVCAFKMASRFPRAYSYWVRYQGPYVSMAFITVLKMVIVVIGMLATGLSPTTRTDPDDPKITIVSCNPNYRNSLLFNTSLDLLLSVVGFSFAYMGKELPTNYNEAKFITLSMTFYFTSSVSLCTFMSAYSGVLVTIVDLLVTVLNLLAISLGYFGPKCYMILFYPERNTPAYFNSMIQGYTMRRD

SEQ ID NO: 14 - DNA, 2559 bp, Homo sapiens
atgctgggccctgctgtcctgggcctcagcctctgggctctcctgcaccctgggacgggggccccattgtgcctgtcacagcaacttaggatgaagggggactacgtgctgggggggctgttccccctgggcgaggccgaggaggctggcctccgcagccggacacggcccagcagccctgtgtgcaccaggttctcctcaaacggcctgctctgggcactggccatgaaaatggccgtggaggagatcaacaacaagtcggatctgctgcccgggctgcgcctgggctacgacctctttgatacgtgctcggagcctgtggtggccatgaagcccagcctcatgttcctggccaaggcaggcagccgcgacatcgccgcctactgcaactacacgcagtaccagccccgtgtgctggctgtcatcgggccccactcgtcagagctcgccatggtcaccggcaagttcttcagcttcttcctcatgccccaggtcagctacggtgctagcatggagctgctgagcgcccgggagaccttcccctccttcttccgcaccgtgcccagcgaccgtgtgcagctgacggccgccgcggagctgctgcaggagttcggctggaactgggtggccgccctgggcagcgacgacgagtacggccggcagggcctgagcatcttctcggccctggccgcggcacgcggcatctgcatcgcgcacgagggcctggtgccgctgccccgtgccgatgactcgcggctggggaaggtgcaggacgtcctgcaccaggtgaaccagagcagcgtgcaggtggtgctgctgttcgcctccgtgcacgccgcccacgccctcttcaactacagcatcagcagcaggctctcgcccaaggtgtgggtggccagcgaggcctggctgacctctgacctggtcatggggctgcccggcatggcccagatgggcacggtgcttggcttcctccagaggggtgcccagctgcacgagttcccccagtacgtgaagacgcacctggccctggccaccgacccggccttctgctctgccctgggcgagagggagcagggtctggaggaggacgtggtgggccagcgctgcccgcagtgtgactgcatcacgctgcagaacgtgagcgcagggctaaatcaccaccagacgttctctgtctacgcagctgtgtatagcgtggcccaggccctgcacaacactcttcagtgcaacgcctcaggctgccccgcgcaggaccccgtgaagccctggcagctcctggagaacatgtacaacctgaccttccacgtgggcgggctgccgctgcggttcgacagcagcggaaacgtggacatggagtacgacctgaagctgtgggtgtggcagggctcagtgcccaggctccacgacgtgggcaggttcaacggcagcctcaggacagagcgcctgaagatccgctggcacacgtctgacaaccagaagcccgtgtcccggtgctcgcggcagtgccaggagggccaggtgcgccgggtcaaggggttccactcctgctgctacgactgtgtggactgcgaggcgggcagctaccggcaaaacccagacgacatcgcctgcaccttttgtggccaggatgagtggtccccggagcgaagcacacgctgcttccgccgcaggtctcggttcctggcatggggcgagccggctgtgctgctgctgctcctgctgctgagcctggcgctgggccttgtgctggctgctttggggctgttcgttcaccatcgggacagcccactggttcaggcctcgggggggcccctggcctgctttggcctggtgtgcctgggcctggtctgcctcagcgtcctcctgttccctggccagcccagccctgcccgatgcctggcccagcagcccttgtcccacctcccgctcacgggctgcctgagcacactcttcctgcaggcggccgagatcttcgtggagtcagaactgcctctgagctgggcagaccggctgagtggctgcctgcgggggccctgggcctggctggtggtgctgctggccatgctggtggaggtcgcactgtgcacctggtacctggtggccttcccgccggaggtggtgacggactggcacatgctgcccacggaggcgctggtgcactgccgcacacgctcctgggtcagcttcggcctagcgcacgccaccaatgccacgctggcctttctctgcttcctgggcactttcctggtgcggagccagccgggccgctacaaccgtgcccgtggcctcacctttgccatgctggcctacttcatcacctgggtctcctttgtgcccctcctggccaatgtgcaggtggtcctcaggcccgccgtgcagatgggcgccctcctgctctgtgtcctgggcatcctggctgccttccacctgcccaggtgttacctgctcatgcggcagccagggctcaacacccccgagttcttcctgggagggggccctggggatgcccaaggccagaatgacgggaacacaggaaatcaggggaaacatgagtga

SEQ ID NO: 15 - protein, 852 aa, Homo sapiens
MLGPAVLGLSLWALLHPGTGAPLCLSQQLRMKGDYVLGGLFPLGEAEEAGLRSRTRPSSPVCTRFSSNGLLWALAMKMAVEEINNKSDLLPGLRLGYDLFDTCSEPVVAMKPSLMFLAKAGSRDIAAYCNYTQYQPRVLAVIGPHSSELAMVTGKFFSFFLMPQVSYGASMELLSARETFPSFFRTVPSDRVQLTAAAELLQEFGWNWVAALGSDDEYGRQGLSIFSALAAARGICIAHEGLVPLPRADDSRLGKVQDVLHQVNQSSVQVVLLFASVHAAHALFNYSISSRLSPKVWVASEAWLTSDLVMGLPGMAQMGTVLGFLQRGAQLHEFPQYVKTHLALATDPAFCSALGEREQGLEEDVVGQRCPQCDCITLQNVSAGLNHHQTFSVYAAVYSVAQALHNTLQCNASGCPAQDPVKPWQLLENMYNLTFHVGGLPLRFDSSGNVDMEYDLKLWVWQGSVPRLHDVGRFNGSLRTERLKIRWHTSDNQKPVSRCSRQCQEGQVRRVKGFHSCCYDCVDCEAGSYRQNPDDIACTFCGQDEWSPERSTRCFRRRSRFLAWGEPAVLLLLLLLSLALGLVLAALGLFVHHRDSPLVQASGGPLACFGLVCLGLVCLSVLLFPGQPSPARCLAQQPLSHLPLTGCLSTLFLQAAEIFVESELPLSWADRLSGCLRGPWAWLVVLLAMLVEVALCTWYLVAFPPEVVTDWHMLPTEALVHCRTRSWVSFGLAHATNATLAFLCFLGTFLVRSQPGRYNRARGLTFAMLAYFITWVSFVPLLANVQVVLRPAVQMGALLLCVLGILAAFHLPRCYLLMRQPGLNTPEFFLGGGPGDAQGQNDGNTGNQGKHE

(19) United States
(12) Patent Application Publication
(10) Pub. No.: US 2013/0040317 A1
(43) Pub. Date: Feb. 14, 2013

(54) CELL LINES COMPRISING ENDOGENOUS TASTE RECEPTORS AND THEIR USES

(71) Applicants: Harish Radhakrishna, Bridgewater, NJ (US); Michael D. Brown, Lilburn, GA (US); David Peter Siderovski, Chapel Hill, NC (US); Adam Kimple, Chapel Hill, NC (US); Staci Padove Cohen, Raleigh, NC (US)

(72) Inventors: Harish Radhakrishna, Bridgewater, NJ (US); Michael D. Brown, Lilburn, GA (US); David Peter Siderovski, Chapel Hill, NC (US); Adam Kimple, Chapel Hill, NC (US); Staci Padove Cohen, Raleigh, NC (US)

(73) Assignees: THE UNIVERSITY OF NORTH CAROLINA AT CHAPEL HILL, Chapel Hill, NC (US); THE COCA-COLA COMPANY, Atlanta, GA (US)

(21) Appl. No.: 13/569,718

(22) Filed: Aug. 8, 2012

Related U.S. Application Data
(60) Provisional application No. 61/521,135, filed on Aug. 8, 2011.

(51) Int. Cl. G01N 33/566; C12N 5/10; G01N 21/55

(52) U.S. Cl. 435/7.21; 435/325

(57) ABSTRACT

Provided herein are cell lines and assays that can be utilized to identify taste receptor modulators.

### CROSS REFERENCE TO RELATED APPLICATIONS

This application claims benefit of and priority to U.S. Provisional Application No. 61/521,135 filed Aug. 8, 2011, the contents of which are hereby incorporated by reference in their entirety.

### REFERENCE TO SEQUENCE LISTING

The Sequence Listing submitted Aug. 8, 2012, as a text file named “10031015US1_ST25.txt,” created on Aug. 7, 2012, and having a size of 34.3 kilobytes is hereby incorporated by reference.

### BACKGROUND

Numerous tastants are utilized in consumables. Additionally, agents can modulate bitter and/or sweet taste, for example, by decreasing bitterness and/or enhancing sweet taste in consumables, such as foods, beverages and medicines. Means for screening agents to identify tastants and to identify modulators of bitter and/or sweet taste receptors are thus useful.

### SUMMARY

This disclosure relates to cell lines and assays that can be utilized to identify taste receptor modulators. For example, provided herein is a method for identifying a bitter taste modulator comprising contacting a cell with a bitter tastant and a test compound, wherein the cell is derived from airway tissue and endogenously expresses a bitter taste receptor and a sweet taste receptor, and measuring bitter taste receptor activity. Optionally, the cell can endogenously express RGS21. A change in bitter taste receptor activity by the bitter tastant in the presence of the test compound indicates modulation of the bitter taste receptor by the test compound, thus identifying a bitter taste modulator.

Further provided is a method for identifying a bitter tastant comprising contacting a cell, wherein the cell is derived from airway tissue and endogenously expresses a bitter taste receptor and a sweet taste receptor, with a test compound and measuring bitter taste receptor activity. Optionally, the cell can endogenously express RGS21. An increase in bitter taste receptor activity indicates that the test compound is a bitter tastant.

Also provided is a method for identifying a sweet taste modulator comprising contacting a cell with a sweetener and a test compound, wherein the cell is derived from airway tissue and endogenously expresses a bitter taste receptor and a sweet taste receptor, and measuring sweet taste receptor activity. Optionally, the cell can endogenously express RGS21. A change in sweet taste receptor activity by the sweetener in the presence of the test compound indicates modulation of the sweet taste receptor by the test compound, thus identifying a sweet taste modulator.

Further provided is a method for identifying a sweetener and/or a bitter tastant comprising contacting a cell, wherein the cell is derived from airway tissue and endogenously expresses a bitter taste receptor and a sweet taste receptor, with a test compound and measuring sweet taste receptor activity. Optionally, the cell can endogenously express RGS21. An increase in sweet taste receptor activity and/or bitter receptor activity indicates that the test compound is a sweetener and/or a bitter tastant.

Also provided is a method for identifying a bitter tastant or modulator comprising contacting a cell with a test compound and measuring bitter taste receptor activity, wherein the cell is a MB9812 cell, a NCI-H520 cell, a NCI-H522 cell or derivative thereof that is derived from airway tissue and endogenously expresses a bitter taste receptor and a sweet taste receptor.

Also provided is a method for identifying a sweet taste modulator or sweetener comprising contacting a cell with a test compound and measuring sweet taste receptor activity and/or bitter taste receptor activity, wherein the cell is a MB9812 cell, a NCI-H520 cell, a NCI-H522 cell or derivative thereof that is derived from airway tissue and endogenously expresses a bitter taste receptor and/or a sweet taste receptor.

Further provided is an isolated, relatively pure population of airway cells that express a sweet taste receptor. Also provided is an isolated, relatively pure population of airway cells that express a bitter taste receptor and a sweet taste receptor. The receptors are optionally endogenously expressed by the airway cell, but the airway cell is, optionally, genetically modified to express one or more bitter taste receptors or to overexpress one or more bitter taste receptors. The airway cell is optionally genetically modified to express one or more sweet taste receptors or to overexpress one or more sweet taste receptors. The airway cell is optionally modified to express or overexpress both sweet and bitter receptors. Optionally, the cell can endogenously express RGS21 but can also be genetically modified to express RGS21 or to overexpress RGS21.

### DESCRIPTION OF DRAWINGS

FIG. 1A shows that MB9812 cells response to a bitter compound, denatonium B, and sweeteners, as demonstrated by an increase in intracellular calcium.

FIG. 1B shows that MB9812 cells respond to a bitter compound, denatonium-B, and sweeteners as measured by FLIPR calcium flux.

FIG. 2A shows that NCI-H520 cells respond to a bitter compound, denatonium-B, and sweeteners, as demonstrated by an increase in intracellular calcium.

FIG. 2B shows that NCI-H520 cells respond to a bitter compound, denatonium-B, and sweeteners, as measured by FLIPR calcium flux.

FIG. 3A shows that NCI-H522 cells respond to bitter compound, denatonium-B, and sweeteners, as demonstrated by an increase in intracellular calcium.

FIG. 3B shows that NCI-H522 cells respond to bitter compound, denatonium-B, and sweeteners, as measured by FLIPR calcium flux.

FIG. 4A shows that the glucose response is effected via a lactisole sensitive receptor in MB9812 cells as evidenced by inhibition of the glucose response by lactisole, a T1R3 inhibitor.

FIG. 4B shows that the sucrose response is effected via a lactisole sensitive receptor in MB9812 cells as evidenced by inhibition of the sucrose response by lactisole, a T1R3 inhibitor.

FIGS. 4C and 4D show that fructose response is effected via a lactisole sensitive receptor in MB9812 cells as evidenced by inhibition of the fructose response by lactisole, a T1R3 inhibitor.

FIG. 5 shows that NCI-H520 cells respond to increasing concentrations of Rebaudioside A, the primary sweetener of Truvia.

### DETAILED DESCRIPTION

### Uses for Cell Lines Comprising Bitter Taste Receptors and Sweet Taste Receptors

Provided herein is a method for identifying a bitter taste modulator comprising contacting a cell with a bitter tastant and a test compound, wherein the cell is derived from airway tissue and endogenously expresses a bitter taste receptor and a sweet taste receptor, and measuring bitter taste receptor activity. Optionally, the cell can endogenously express RGS21. A change in bitter taste receptor activity by the bitter tastant in the presence of the test compound indicates modulation of the bitter taste receptor by the test compound, thus identifying a bitter taste modulator.

As used throughout, a bitter taste modulator is a compound that modulates bitter taste receptor activity, for example, by inhibiting or blocking bitter taste receptor activation by a bitter tastant, or by enhancing bitter taste receptor activation by a bitter tastant. In one example, the methods of identifying bitter taste modulators identify compounds that modulate, preferably block or inhibit, the activation of a bitter taste receptor by a bitter tastant. As used throughout, such blockers or inhibitors act directly on the receptor but can optionally act upstream or downstream of the receptor.

Any cell derived from airway tissue that endogenously expresses a bitter taste receptor and a sweet receptor can be utilized in the methods set forth herein. For example, lung or bronchial cells, such as lung or bronchial epithelial cells can be utilized. Known human airway cell lines can optionally be utilized. Examples of airway cells that can be utilized include, but are not limited to, MB9812 cells, NCI-H520 cells NCI-H522 cells or derivatives thereof, wherein the cells express a bitter taste receptor and a sweet taste receptor.

In the methods set forth herein, the bitter taste receptor responds to at least one bitter tastant or bitterant. Bitter tastants include, but are not limited to, acesulfame K, acetaminophen, 2-acetyl pyrazine, aloin, amino-2-norbornane-carboxylic acid, amygadalin, andrographolide, arbutin, aristolochic acid, atropine, brucine, 4-benzylpiperidine, caffeine, chloramphenicol, chloroquine, cinchonine, ciprofloxacin, clarithromycin, clindamycin, cycloheximide, cyclooctanone, denatonium benzoate, dexamethasone, diltiazem hydrochloride, diisobutylamine, dimethylbiguanide, 2,6-dimethylpiperidine, doxepin, enalapril maleate, edrophonium, enoxacin, (−)-epicatechin, (−)-erythromycin, ethylpyrazine, famotidine, gabapentin, ginkgolide A, goitrin, guaicol glyceryl ether, labetalol-HCl, linamarin, lomefloxacin, (−)-lupinine, N-methylthiourea, 1-methy-2-quinolinone, methylprednisolone, nitrophthalene, nitrosaccharin, ofloxacin, oleuropein, omeprazole, oxybutynin chloride, oxyphenonium HBr, peptide-LPFNQL (SEQ ID NO: 1), peptide-LPFSQL (SEQ ID NO: 2), peptide-YQEPVLGPVRGPFPIIV (SEQ ID NO: 2), peptide-PVLGPVRGPFPIIV (SEQ ID NO: 3), peptide-PVRGPFPIIV (SEQ ID NO: 4), peptide-RGPFPIIV (SEQ ID NO: 5), N-ethyl-N′-phenylurea, 2-picoline, picric acid, pirenzepine dihydrochloride, phenylthiocarbamide, prednisone, procainamide, 6-n-propyl-2-thiouracil, quassin, quinacrine, quinine, ranitidine, saccharin, D-(−)-salicin, spartein sulfate pentahydrate, sucrose octaacetate, strychnine, sulfamethoxazole, theobromine, thioacetanilide, thiocarbanilide, tolazoline, tolylurea, trapidil, trimethoprim, and L-tryptophan.

As used throughout, a test compound can be a naturally occurring compound, a protein, a peptide, a polysaccharide, a chemical, a small molecule or a polynucleotide (for example, a cDNA, an aptamer, a morpholino, a triple helix molecule, an siRNA, a shRNA, an miRNA, an antisense RNA, an LNA, a ribozyme or any other polynucleotide now known or identified in the future). In the methods set forth herein, the compound can be in a library. The libraries can comprise natural products or synthetic compounds. Therefore, provided herein are methods for screening libraries of compounds in order to identify a bitter taste modulator or a bitter tastant. RGS21 is also known as a regulator of G-protein signaling 21 and is capable of binding to or inhibiting Gαi class proteins or other Gα proteins. As set forth above, the airway cells utilized in the present methods can optionally endogenously express RGS21. RGS21 can be encoded by a nucleotide sequence comprising the human sequence set forth in GenBank Accession No. AY643711.1 (SEQ ID NO: 6). This nucleotide sequence encodes the protein sequence set forth in GenBank Accession No. NP_{—}001034241.1 (SEQ ID NO: 7). Airway cells from human or other species comprising an RGS21 nucleotide sequence or an RGS21 protein sequence that is at least 60%, 65%, 70%, 75%, 80%, 85%, 90%, 95%, 95%, 97%, 98%, 99% or more identical to the sequence set forth in GenBank Accession No. AY643711.1 or the sequence set forth in GenBank Accession No. NP_{—}001034241.1, respectively, can also be utilized in the methods described herein. Optionally, the protein sequence comprises one or more conservative amino acid substitutions as compared to the provided sequence. In particular, cells comprising an RGS21 sequence, wherein the RGS21 retains at least one activity of RGS21, for example, interaction with a Ga protein can be utilized in the methods set forth herein.

The cells described herein can be genetically modified to express or overexpress RGS21. For example, an airway cell described herein can be genetically modified by introducing an exogenous nucleic acid comprising a nucleotide sequence encoding RGS21. The nucleic acid can be stably or transiently introduced into the cell. A cell that is genetically modified includes a cell wherein the introduced nucleic acid is also endogenous to the cell. The exogenous nucleic acid can be in a construct or vector that comprises a promoter that is operably linked to the nucleotide sequence encoding RGS21. The promoter can be a constitutive promoter or an inducible promoter. Exemplary inducible promoters include tissue-specific promoters and promoters responsive or unresponsive to a particular stimulus (such as light, oxygen or chemical concentration, for example, for a tetracycline inducible promoter).

As utilized throughout, Gα proteins include all members of the Gαi class now known or later discovered, including but not limited to, Gαi1, Gαi2, and Gαi3, gustducin, transducin, Gαo, Gαtr, Gαg, Gαtr, Gαtc and Gαz. Also included are all members of the Gq class now known or later discovered, including, but not limited to, Gαq Gαl1, Gαl4, Gαl5 and Gαl6. The cells described herein can comprise one or more types of Gα protein that are endogenously or recombinantly expressed in the cells. The cells can also comprise chimeric Gα proteins, for example, Gαq-Gustducin or Gα16-gustducin 44, as described in U.S. Patent Publication No. 20090311686, incorporated herein in its entirety by this reference.

The bitter taste receptor can be selected from any bitter taste receptor, including, for example, T2R46 or T2R38. T2R46 is also known as taste receptor type 2, member 46 of the G protein-coupled receptor family and mediates the perception of bitterness through a G protein-coupled second messenger pathway. An example of a nucleotide sequence encoding T2R46 is the human sequence set forth in GenBank Accession No. NM_{—}176887.2 (SEQ ID NO: 8). This sequence encodes the protein sequence set forth in GenBank Accession No. NP_{—}795368.2 (SEQ ID NO: 9). Airway cells from human or other species endogenously comprising a T2R46 nucleotide sequence or a T2R46 protein sequence that is at least 60%, 65%, 70%, 75%, 80%, 85%, 90%, 95%, 95%, 97%, 98%, 99% or more identical to the sequence set forth in GenBank Accession No. NM 176887.2, or GenBank Accession No. NP_{—}795368.2 can be utilized in the methods set forth herein. Optionally, the protein sequence comprises one or more conservative amino acid substitutions as compared to the provided sequence. In particular, cells comprising a T2R46 sequence, wherein the T2R46 receptor retains the ability to respond to at least one bitter tastant, can be used in the methods described herein.

T2R38 is also known as taste receptor type 2, member 38 of the G protein-coupled receptor family and also mediates the perception of bitterness through a G protein-coupled second messenger pathway. An example of a nucleotide sequence encoding T2R38 is the human sequence set forth in GenBank Accession No. NM 176817.4 (SEQ ID NO: 10). This sequence encodes the protein sequences set forth in GenBank Accession No. NP_{—}789787.4 (SEQ ID NO: 11). Airway cells from human or other species endogenously comprising a T2R38 nucleotide sequence or a T2R38 protein sequence that is at least 60%, 65%, 70%, 75%, 80%, 85%, 90%, 95%, 95%, 97%, 98%, 99% or more identical to the sequence set forth in GenBank Accession No. NM_{—}176817.4 or GenBank Accession No. NP_{—}789787.4, can be utilized in the methods set forth herein. Optionally, the protein sequence comprises one or more conservative amino acid substitutions as compared to the provided sequence. In particular, cells comprising a T2R38 sequence, wherein the T2R38 receptor retains the ability to respond to at least one bitter tastant, can be used in the methods described herein.

The cells described herein can be genetically modified to express or overexpress the bitter taste receptor. For example, an airway cell described herein can be genetically modified by introducing an exogenous nucleic acid comprising a nucleotide sequence encoding T2R46 or T2R38. The nucleic acid can be stably or transiently introduced into the cell. A cell that is genetically modified includes a cell wherein the introduced nucleic acid is also endogenous to the cell. The exogenous nucleic acid can be in a construct or vector that comprises a promoter that is operably linked to the nucleotide sequence encoding T2R46 or T2R38. The promoter can be a constitutive promoter or an inducible promoter. Exemplary inducible promoters include tissue-specific promoters and promoters responsive or unresponsive to a particular stimulus (such as light, oxygen or chemical concentration, for example, a tetracycline inducible promoter).

In the methods described herein, the cell(s) can be grown on an appropriate substrate, such as a multi-well plate, a tissue culture dish, a flask, etc. The cell can be in a population of cells. This population can be an isolated, relatively pure population of airway cells. One of skill in the art would know how to select the appropriate growth conditions and medium for a given cell type. The methods described herein can further comprise contacting the cell with a dye, substrate, assay medium or any other composition necessary to assess the output from a signaling pathway. For example, the method can comprise loading the cells with calcium-sensitive fluorescent dye in order to measure changes in cytoplasmic calcium levels. The incubation periods necessary to effect bitter taste activation and subsequent assessment of bitter taste receptor activity will vary by cell type but can be empirically determined by one of skill in the art. The cell(s) can be contacted with a test compound before, during or after contacting the cells with the bitter tastant. Screening methods can optionally be performed in vivo. Therefore, the cell can be in a subject.

In the methods described throughout, taste receptor activity can be measured by any means standard in the art. Any suitable physiological change that is a consequence of G protein-coupled receptor activity can be used to assess the effect of a test compound on a taste receptor. Methods for assaying G protein coupled receptor activity are available in the art (see Williams and Hill “GPCR signaling: understanding the pathway to successful drug discovery,” Methods Mol Biol. 2009;552:39-50 (2009); and De los Frailes and Diez “Screening technologies for G protein-coupled receptors: from HTS to uHTS,” Methods Mol Biol. 552:15-37 (2009)).

One of skill in the art can measure changes in the level of a second messenger in the cell. Examples of second messengers include, cAMP, cGMP, diacylglycerol (DAG), Phosphatidylinositol 4,5-bisphosphate (PIP2), inositol 1,4,5-trisphosphate (IP₃) and intracellular calcium. For example, changes in intracellular cAMP or cGMP can be measured using immunoassays. The method described in Offermanns & Simon, J. Bio. Chem., 270:15175-15180 (1995), can be used to determine the level of cAMP. Also, the method described in Felley-Bosco et al., Am. J. Resp. Cell and Mol. Biol., 11:159-164 (1994), can be used to determine the level of cGMP. Further, an assay kit for measuring cAMP and/or cGMP is described in U.S. Pat. No. 4,115,538, incorporated herein by this reference.

Activation of some G protein-coupled receptors stimulates the formation of inositol triphosphate (IP₃) through phospholipase C-mediated hydrolysis of phosphatidylinositol. IP₃ stimulates the release of intracellular calcium ions. Thus, a change in cytoplasmic calcium ion levels, or a change in second messenger levels, such as IP₃ can be used to assess G protein-coupled receptor function. Increased cytoplasmic calcium levels can result from the release of intracellular calcium stores as well as from extracellular calcium entry via plasma membrane ion channels. Methods for measuring changes in cytoplasmic calcium levels are available to those of skill in the art. For example, calcium levels can be measured using fluorescent Ca²⁺ indicator dyes and fluorimetric imaging (See Liu et al. “A multiplex calcium assay for identification of GPCR agonists and antagonists,” Assay Drug Dev Technol. June; 8(3):367-79 (2010); and Liu et al. “Comparison on functional assays for Gq-coupled GPCRs by measuring inositol monophospate-1 and intracellular calcium in 1536-well plate format,” Curr Chem Genomics. 2008 Jul. 11; 1:70-8 (2008)).

RGS21 GTPase activating protein (GAP) activity can also be measured to assess receptor activity. For example, one of skill in the art can measure a change in the interaction between RGS21 and a G protein, for example, a Gα protein. This interaction can be measured by fluorescence resonance energy transfer, immunoassay or any other means for measuring the interaction between two proteins. Also, radiolabelled (or fluorescent) GTPγS binding to isolated membrane preps from cells expressing the appropriate endogenous tastant receptor can be measured (See, for example, Cooper et al. “[35S]GTPgammaS binding G protein-coupled receptor assays” Methods Mol. Biol. 552:143-151 (2009)). In these methods, activation of the receptor leads to guanine nucleotide exchange on the heterotrimeric G-protein, leading the G-alpha subunit to bind (irreversibly) to the radiolabeled (or fluorescent) GTPyS.

Binding activity can also be used to measure taste receptor activity, for example, via competitive binding assay or surface plasmon resonance (see Salamon et al. “Chapter 6. Plasmon resonance methods in membrane protein biology applications to GPCR signaling,” Methods Enzymol. 2009; 461:123-46 (2009); and Harding et al. “Direct analysis of a GPCR-agonist interaction by surface plasmon resonance,” Eur Biophys J. October; 35(8):709-12 (2006)).

Receptor internalization and/or receptor desensitization can also be measured (see, for example, Kershaw et al. “Analysis of chemokine receptor endocytosis and intracellular trafficking,” Methods Enzymol. 460:357-77(2009); and Di Certo et al. “Delayed internalization and lack of recycling in a beta2-adrenergic receptor fused to the G protein alpha-subunit,” BMC Cell Biol. October 7; 9:56(2008)). Receptor-dependent activation of gene transcription can also be measured to assess taste receptor activity. The amount of transcription may be measured by using any method known to those of skill in the art. For example, mRNA expression of the protein of interest may be detected using PCR techniques, microarray or Northern blot. The amount of a polypeptide produced by an mRNA can be determined by methods standard in the art for quantitating proteins in a cell, such as Western blotting, ELISA, ELISPOT, immunoprecipitation, immunofluorescence (e.g., FACS), immunohistochemistry, immunocytochemistry, etc., as well as any other method now known or later developed for quantitating protein in or produced by a cell.

Beta-arrestin recruitment and/or receptor desensitization is optionally measured. See, for example, Bohn et al., “Seeking Ligand Bias: Assessing GPCR Coupling to Beta-Arrestins for Drug Discovery. Drug Discov Today Technol. Spring;7(1):e37-e42 (2010).

Taste receptor dependent physical changes to a cell can also be measured, for example, by microscopically assessing size, shape, density or any other physical change mediated by taste receptor activation. Flow cytometry can also be utilized to assess physical changes and/or determine the presence or absence of cellular markers.

This method can further comprise contacting the cell with a second bitter tastant, after contacting the cell with the test compound and the first bitter tastant and prior to measuring bitter taste receptor activity. The first bitter tastant and the second bitter tastant can be the same or different.

When measuring a change in bitter taste receptor activity, bitter receptor activity in a cell contacted with a test compound and a bitter tastant can be compared to bitter receptor activity in a control cell contacted with a bitter tastant, but not contacted with the test compound. Bitter taste receptor activity can also be compared to bitter taste receptor activity in the same cell prior to addition of the test compound or after the effect of the test compound has subsided. For example, decreased concentration of cAMP can occur upon bitter receptor activation. If an increase in cAMP concentration is measured in a cell contacted with a test compound and a bitter tastant as compared to a cell contacted with the bitter tastant, the test compound is a bitter taste modulator that inhibits activation of a bitter taste receptor by the bitter tastant. If a decrease in cAMP concentration is measured in a cell contacted with a test compound and a bitter tastant as compared to a cell contacted with the bitter tastant, the test compound is a bitter taste modulator that enhances activation of a bitter taste receptor by the bitter tastant. In another example, increased release of intracellular calcium can occur upon bitter receptor activation. If a decrease in intracellular calcium is measured in a cell contacted with a test compound and a bitter tastant as compared to a cell contacted with the bitter tastant, the test compound is a bitter taste modulator that inhibits activation of a bitter taste receptor by the bitter tastant. If an increase in intracellular concentration is measured in a cell contacted with a test compound and a bitter tastant as compared to a cell contacted with the bitter tastant, the test compound is a bitter taste modulator that enhances activation of a bitter taste receptor by the bitter tastant. These examples are merely exemplary as any parameter described herein can be measured and compared to appropriate control cells to measure changes in bitter taste receptor activity effected by test compounds.

This method can further comprise measuring the effect of the identified bitter taste modulator in a human or other taste tests in order to evaluate the effect of the bitter taste modulator on bitter taste. Any of the bitter taste modulators identified via the methods described herein can be used in foods, beverages and medicines as flavor or taste modulators in order to inhibit the bitter taste associated with beverages, foods or medicines.

As utilized throughout, consumables include all food products, including but not limited to, cereal products, rice products, tapioca products, sago products, baker's products, biscuit products, pastry products, bread products, confectionery products, dessert products, gums, chewing gums, chocolates, ices, honey products, treacle products, yeast products, baking-powder, salt and spice products, savory products, mustard products, vinegar products, sauces (condiments), tobacco products, cigars, cigarettes, processed foods, cooked fruits and vegetable products, meat and meat products, jellies, jams, fruit sauces, egg products, milk and dairy products, yoghurts, cheese products, butter and butter substitute products, milk substitute products, soy products, edible oils and fat products, medicaments, beverages, carbonated beverages, alcoholic drinks, beers, soft drinks, mineral and aerated waters and other non-alcoholic drinks, fruit drinks, fruit juices, coffee, artificial coffee, tea, cocoa, including forms requiring reconstitution, food extracts, plant extracts, meat extracts, condiments, sweeteners, nutraceuticals, gelatins, pharmaceutical and non-pharmaceutical gums, tablets, lozenges, drops, emulsions, elixirs, syrups and other preparations for making beverages, and combinations thereof.

This method can further comprise comparing bitter receptor activity in a cell contacted with an identified bitter taste modulator and a known bitter tastant with bitter receptor activity in a control cell contacted with a known bitter taste modulator and a known bitter tastant. One of skill in the art would know that known bitter taste modulators have established potencies or activity levels. By comparing bitter taste modulators identified by the methods described herein with known bitter taste modulators, potencies can be established for the identified bitter taste modulators. Depending on the amount of bitter taste receptor activity necessary for a particular food, beverage, medicine or process, one of skill in the art can select one or more of the bitter taste modulators identified by the methods set forth herein based on its potency. The bitter taste modulators identified by the methods set forth herein can be combined with known bitter tastants, sweeteners, umami tastants, bitter taste modulators, sweet taste modulators, umami taste modulators or any combination thereof.

Further provided is a method for identifying a bitter tastant comprising contacting a cell, wherein the cell is derived from airway tissue and endogenously expresses a bitter taste receptor and a sweet receptor with a test compound, and measuring bitter taste receptor activity. Optionally, the cell endogenously expresses RGS21. An increase in bitter taste receptor activity indicates that the test compound is a bitter tastant.

When measuring bitter taste receptor activity, bitter receptor activity in a cell contacted with a test compound can be compared to bitter receptor activity in a control cell not contacted with the test compound. Bitter taste receptor activity can also be compared to bitter taste receptor activity in the same cell prior to addition of the test compound or after the effect of the test compound has subsided. For example, decreased concentration of cAMP can occur upon bitter receptor activation. If a decrease in cAMP concentration is measured in a cell contacted with a test compound as compared to a control cell not contacted with the test compound, the test compound is a bitter tastant. In another example, increased release of intracellular calcium can occur upon bitter receptor activation. If an increase in intracellular calcium concentration is measured in a cell contacted with a test compound as compared to a control cell not contacted with the test compound, the test compound is a bitter tastant. These examples are merely exemplary as any parameter described herein can be measured and compared to appropriate control cells to measure bitter taste receptor activity effected by test compounds.

This method can further comprise measuring the effect of the identified bitter tastant in a human or other taste tests in order to evaluate the effect of the bitter tastant on bitter taste. Any of the bitter tastants identified via the methods described herein can be used in consumables such as foods, beverages and medicines in order to increase bitterness associated with beverages, foods or medicines. Alternatively, any of the bitter tastants identified via the methods described herein can be selectively removed from beverages, foods or medicines or the processes utilized to make beverages, food and medicines in order to reduce bitterness.

This method can further comprise comparing bitter receptor activity in a cell contacted with an identified bitter tastant with bitter receptor activity in a control cell contacted with a known bitter tastant. One of skill in the art would know that known bitter tastants have established potencies or activity levels. By comparing bitter tastants identified by the methods described herein with known bitter tastants, potencies can be established for the identified bitter tastants. Depending on the amount of bitter taste receptor activity necessary for a particular food, beverage, medicine or process, one of skill in the art can select one or more of the bitter tastants identified by the methods set forth herein based on its potency. The bitter tastants identified by the methods set forth herein can be combined with known bitter tastants, sweeteners or umami tastants.

Cell lines and methods similar to those described above can be used related to sweeteners and modulators thereof alone or in combination with bitter tastants and modulators thereof. Thus, provided is a method for identifying a sweet taste modulator comprising contacting a cell with a sweetener and a test compound, wherein the cell is derived from airway tissue and endogenously expresses a bitter taste receptor and a sweet taste receptor, and measuring sweet taste receptor activity. Optionally, the cell can endogenously express RGS21. A change in sweet taste receptor activity by the sweetener in the presence of the test compound indicates modulation of the sweet taste receptor by the test compound, thus identifying a sweet taste modulator. Optionally, a bitter taste modulator is screened concurrently as set out above.

Also provided is a method for identifying a sweet taste modulator comprising contacting a cell with a sweetener and a test compound, wherein the cell is derived from airway tissue and endogenously expresses a sweet taste receptor and measuring sweet taste receptor activity. The cell can optionally endogenously express RGS21. A change in sweet taste receptor activity by the sweetener indicates modulation of the sweet taste receptor by the test compound, thus identifying a sweet taste modulator.

As used throughout, a sweet taste modulator is a compound that modulates sweet taste receptor activity, for example, by inhibiting or blocking sweet taste receptor activation by a sweetener, or by enhancing sweet taste receptor activation by a sweetener. In one example, the methods of identifying sweet taste modulators identify compounds that modulate, preferably enhance, the activation of a sweet taste receptor by a sweetener. As described for methods related to screening for modulators of bitter taste, such modulators can act directly on the receptor or upstream or downstream of the receptor.

Any cell derived from airway tissue that endogenously expresses a sweet taste receptor can be utilized in the methods set forth herein. Optionally, the cells can endogenously express RGS21. For example, lung or bronchial cells, such as lung or bronchial epithelial cells can be utilized. Known human airway cell lines can optionally be utilized. Examples of airway cells that can be utilized include, but are not limited to, MB9812 cells, NCI-H520 cells, NCI-H522 cells or derivatives thereof, wherein the cells express a sweet taste receptor and, optionally, a bitter taste receptor.

In the methods set forth herein, the sweet taste receptor responds to at least one sweetener. The sweetener can be an artificial sweetener or a natural sugar. For example, the sweetener can be a carbohydrate sweetener, including but not limited to sucrose, glucose, fructose, HFCS, HFSS, D-Tagatose, Trehalose, D-galactose, Rhamnose. The sweetener can also be a synthetic high-potency sweeteners, including but not limited to, aspartame, neotame, acesulfame K, sucralose, cyclamate, saccharin, neohesperidindihydrochalcone. The sweetener can also be a natural high-potency sweetener, including but not limited to, rebaudioside A, Rebaudioside B, Rebaudioside C, Rebaudioside D, Rebaudioside E, Dulcoside A, Dulcoside B, Rubusoside, Stevioside, Mogroside IV, Mogroside V, Monatin, Curculin, Glycyrrhizin, Thaumatin, Monellin, Mabinlin, Brazzein, Monatin, Hernandulcin, Phyllodulci. Also included are polyols such as, Erythritol, Maltitol, Mannitol, Sorbitol, Lactitol, Xylitol, Isomalt, and amino acids, including but not limited to, glycine, D- or L-alanine, D-tryptophan, arginine, serine and threonine.

The sweet taste receptor can be T1R2/T1R3. T1R2/T1R3 is a heterodimer comprising T1R2, also known as taste receptor type 1, member 2, and T1R3, also known as taste receptor type 1, member 3. This receptor mediates the perception of sweet taste through a G protein-coupled second messenger pathway. An example of a nucleotide sequence encoding T1R2 is the human sequence set forth in GenBank Accession No. NM_{—}152232.2 (SEQ ID NO: 12). This sequence encodes the protein sequence set forth in GenBank Accession No. NP_{—}689418.2 (SEQ ID NO: 13). An example of a nucleotide sequence encoding T1R3 is the human sequence set forth in GenBank Accession No. NM_{—}152228.1 (SEQ ID NO: 14). This sequence encodes the protein sequence set forth in GenBank Accession No. NP_{—}689414.1 (SEQ ID NO: 15). Airway cells from human or other species endogenously comprising a T1R2/T1R3 nucleotide sequence or a T1R2/T1R3 protein sequence that is at least 60%, 65%, 70%, 75%, 80%, 85%, 90%, 95%, 95%, 97%, 98%, 99% or more identical to the sequence set forth in GenBank Accession Nos. NM_{—}152232.2/NM 152228.1, or GenBank Accession Nos. NP_{—}689418.2/NP_{—}689414.1, can be utilized in the methods set forth herein. Optionally, the protein sequence comprises one or more conservative amino acid substitutions as compared to the provided sequence. In particular, cells comprising a T1R2/T1R3 sequence, wherein the T1R2/T1R3 receptor retains the ability to respond to at least one sweetener, can be used in the methods described herein.

The cells described herein can be modified to express or overexpress a sweet taste receptor and, optionally, a bitter receptor as well. For example, an airway cell described herein can be genetically modified by introducing an exogenous nucleic acid comprising a nucleotide sequence encoding T1R2/T1R3. The nucleic acid can be stably or transiently introduced into the cell. A cell that is genetically modified includes a cell wherein the introduced nucleic acid is also endogenous to the cell. The exogenous nucleic acid can be in a construct or vector that comprises a promoter that is operably linked to the nucleotide sequence encoding T1R2/T1R3. The nucleotide sequence for T1R2 and the nucleotide sequence for T1R3 can be in the same construct or in separate constructs. Also provided is an airway cell that endogenously expresses T1R2, wherein a nucleotide sequence encoding T1R3 is exogenously introduced into the cell. Also provided is an airway cell that endogenously expresses T1R3, wherein a nucleotide sequence encoding T1R2 is exogenously introduced into the cell. Also provided is an airway cell that endogenously expresses RGS21 and T1R2, wherein a nucleotide sequence encoding T1R3 is exogenously introduced into the cell. Also provided is an airway cell that endogenously expresses RGS21 and T1R3, wherein a nucleotide sequence encoding T1R2 is exogenously introduced into the cell. The promoter can be a constitutive promoter or an inducible promoter. Exemplary inducible promoters include tissue-specific promoters and promoters responsive or unresponsive to a particular stimulus (such as light, oxygen or chemical concentration, for example, for a tetracycline inducible promoter).

Methods for measuring taste receptor activity are described above. When measuring a change in measuring sweet taste receptor activity, sweet receptor activity in a cell contacted with a test compound and a sweetener can be compared to sweet receptor activity in a control cell contacted with a sweetener, but not contacted with the test compound. Sweet taste receptor activity can also be compared to sweet taste receptor activity in the same cell prior to addition of the test compound or after the effect of the test compound has subsided.

For example, increased intracellular concentration of cAMP can occur upon sweet receptor activation. If a decrease in intracellular cAMP concentration is measured in a cell contacted with a test compound and a sweetener as compared to a cell contacted with the sweetener, the test compound is a sweet taste modulator that inhibits activation of a sweet taste receptor by the sweetener. If an increase in intracellular cAMP concentration is measured in a cell contacted with a test compound and a sweetener as compared to a cell contacted with the sweetener, the test compound is a sweet taste modulator that enhances activation of a sweet taste receptor by the sweetener. Sweetness enhancers are useful in the food and flavor industry. Their use allows reduction of the level of sweeteners, including sugars and artificial sweeteners, in consumable products. The use of sweetness enhancers can also reduce calories, prevent tooth decay, and reduce aftertastes. The use of sweetness enhancers in medicaments can also increase patient compliance with oral pharmaceuticals and nutraceuticals.

In another example, increased release of intracellular calcium can occur upon sweet receptor activation. If a decrease in intracellular calcium is measured in a cell contacted with a test compound and a sweetener as compared to a cell contacted with the sweetener alone, the test compound is a sweet taste modulator that inhibits activation of a sweet taste receptor by the sweetener. If an increase in intracellular calcium concentration is measured in a cell contacted with a test compound and a sweetener as compared to a cell contacted with the sweetener alone, the test compound is a sweet taste modulator that enhances activation of a sweet taste receptor by the sweetener. These examples are merely exemplary as one of skill in the art would know that any parameter described herein can be measured and compared to appropriate control cells to measure changes in sweet taste receptor activity effected by test compounds.

This method can further comprise contacting the cell with a second sweetener, after contacting the cell with the test compound and the first sweetener, prior to measuring sweet taste receptor activity. The first sweetener and the second sweetener can be the same or different.

This method can further comprise measuring the effect of the identified sweet taste modulator in a human or other taste tests in order to evaluate the effect of the sweet taste modulator on sweet taste. Any of the sweet taste modulators identified via the methods described herein can be used in consumables as flavor or taste modulators in order to inhibit or enhance sweet taste associated with beverages, foods or medicines.

This method can further comprise comparing sweet receptor activity in a cell contacted with an identified sweet taste modulator and a known sweetener with sweet receptor activity in a control cell contacted with a known sweet taste modulator and a known sweetener. One of skill in the art would know that known sweet taste modulators have established potencies or activity levels. By comparing sweet taste modulators identified by the methods described herein with known sweet taste modulators, potencies can be established for the identified sweet taste modulators. Depending on the amount of sweet taste receptor activity necessary for a particular food, beverage, medicine or process, one of skill in the art can select one or more of the sweet taste modulators identified by the methods set forth herein based on its potency. The sweet taste modulators identified by the methods set forth herein can be combined with known bitter tastants, sweeteners, umami tastants, bitter taste modulators, sweet taste modulators, umami taste modulators or any combination thereof.

Further provided is a method for identifying a sweetener comprising, contacting a cell, wherein the cell is derived from airway tissue and endogenously expresses a sweet taste receptor and a bitter receptor, with a test compound and measuring sweet taste receptor activity. The cell can optionally endogenously express RGS21. An increase in sweet taste receptor activity indicates that the test compound is a sweetener.

Also provided is a method for identifying a sweetener comprising, contacting a cell, wherein the cell is derived from airway tissue and endogenously expresses a sweet taste receptor with a test compound and measuring sweet taste receptor activity. The cell can optionally endogenously express RGS21. An increase in sweet taste receptor activity indicates that the test compound is a sweetener.

When measuring sweet taste receptor activity, sweet receptor activity in a cell contacted with a test compound can be compared to sweet receptor activity in a control cell not contacted with the test compound. Sweet taste receptor activity can also be compared to sweet taste receptor activity in the same cell prior to addition of the test compound or after the effect of the test compound has subsided. For example, increased concentration of cAMP can occur upon sweet receptor activation. If an increase in intracellular cAMP concentration is measured in a cell contacted with a test compound as compared to a control cell not contacted with the test compound, the test compound is a sweetener. In another example, increased release of intracellular calcium can occur upon sweet receptor activation. If an increase in intracellular calcium concentration is measured in a cell contacted with a test compound as compared to a control cell not contacted with the test compound, the test compound is a sweetener. These examples are merely exemplary as any parameter described herein can be measured and compared to appropriate control cells to measure sweet taste receptor activity effected by test compounds.

This method can further comprise measuring the effect of the identified sweetener in a human or other taste tests in order to evaluate the effect of the sweetener on sweet taste. Any of the sweeteners identified via the methods described herein can be used in foods, beverages and medicines in order to increase the sweet taste associated with beverages, foods or medicines. Alternatively, any of the sweeteners identified via the methods described herein can be selectively removed from specific beverage, foods or medicines in order to reduce the sweet taste associated with beverages, foods or medicines.

This method can further comprise comparing sweet receptor activity in a cell contacted with an identified sweetener with sweet receptor activity in a control cell contacted with a known sweetener. One of skill in the art would know that known sweeteners have established potencies or activity levels. By comparing sweeteners identified by the methods described herein with known sweeteners, potencies can be established for the identified sweeteners. Depending on the amount of sweet taste necessary for a particular food, beverage, medicine or process, one of skill in the art can select one or more of the sweeteners identified by the methods set forth herein based on its potency.

### Cell Lines

Provided herein is an isolated, relatively pure population of airway cells that express a sweet taste receptor and, optionally, a bitter taste receptor as well. The sweet taste receptor can be T1R2/T1R3. The cells can optionally endogenously express RGS21.

Further provided is an isolated, relatively pure population of airway cells that express a bitter taste receptor and a sweet taste receptor. The bitter taste receptor can be T2R46 or T2R38. The sweet taste receptor can be T1R2/T1R3. Optionally, the cells endogenously express the bitter taste receptor and RGS21.

Also provided is a panel of cells that can be utilized to assess bitter taste receptor activity and sweet taste receptor activity for a test compound. For example, a first airway cell that endogenously expresses a bitter taste receptor and a sweet taste receptor can be contacted with the test compound and a second airway cell that endogenously expresses a bitter taste receptor and sweet taste receptor can be contacted with the test compound. The first and the second airway cells can be from the same cell line or from different cell lines. Taste receptor activity can be measured in each cell as described herein. The test compound can then be identified as a bitter tastant or a sweetener. If the test compound is identified as a bitter tastant or a sweetener, subsequent tests can be performed with cell populations expressing only one type of receptor (for example a cell population that expresses only a bitter receptor or a cell population that expresses only a sweet receptor) for more specific analysis.

Similarly, a first airway cell that endogenously expresses a bitter taste receptor and a sweet receptor can be contacted with a bitter tastant and the test compound and a second airway cell that endogenously expresses a bitter receptor and a sweet taste receptor can be contacted with a sweetener and the test compound. Taste receptor activity can be measured in each cell as described herein. The test compound can be then be identified as a bitter taste modulator and/or a sweet taste modulator.

For example, a panel of MB9812 and NCI-H520 cells can be utilized to assess bitter taste receptor activity and sweet taste receptor activity. In another example, a panel of MB9812 and NCI-H522 cells can be utilized to assess bitter taste receptor activity and sweet taste receptor activity. In another example, a panel comprising a first population of MB9812 cells and a second population of MB982 cells can be utilized to assess bitter taste receptor activity and sweet taste receptor activity. These examples are not meant to be limiting, as a panel of cells can comprise any airway cell that endogenously expresses a sweet receptor and a bitter receptor.

As used herein, the terms isolated and relatively pure refer to a state of purification greater than that which occurs naturally. In particular, isolated populations of cells described herein are substantially free from the materials with which the cells are normally associated in nature. By relatively pure is meant in a percentage of purity that exceeds nature, including for example 80% to 100% pure or any value in between.

As used in the specification and the appended claims, the singular forms “a, an, and the” include plural referents unless the context clearly dictates otherwise. The term or refers to a single element of stated alternative elements or a combination of two or more elements, unless the context clearly indicates otherwise. As used herein, comprises means includes. Thus, comprising A or B, means “including A, B, or A and B, without excluding additional elements.

The following examples are put forth so as to provide those of ordinary skill in the art with a complete disclosure and description of how the compounds and/or methods claimed herein are made and evaluated, and are intended to be purely exemplary of the invention and are not intended to limit the scope of what the inventors regard as their invention except as and to the extent that they are included in the accompanying claims. Efforts have been made to ensure accuracy with respect to numbers (e.g., amounts, temperature, etc.), but some errors and deviations should be accounted for.

### EXAMPLES

Using standard RT-PCR techniques, cell lines were selected that endogenously express a sweet taste receptor. These cells lines include, but are not limited to, MB9812, NCI-H520 and NCI-H522. MB9812, NCI-H520 and NCI-H522 express RGS21 as well as sweet taste receptor T1R2/T1R3. MB9812, NCI-H520 and NCI-H522 cells also express bitter taste receptors T2R46 and T2R38.

### Functional Assay Using Tastants

### Fluorescence Imaging Plate Reader (FLIPR) Calcium Flux Assays

Calcium flux assays were performed as previously described in Strachan et al., “Ribosomal S6 kinase 2 directly phosphorylates the 5-hydroxytryptamine 2A (5-HT2A) serotonin receptor, thereby modulating 5-HT2A signaling,” J Biol Chem 284:5557-5573 (2009). MB9812, NCI-H520 or NCI-H522 cells were trypsinized, counted, and seeded onto clear-bottomed 96 well plates (Greiner Bio-One; Monroe, N.C.) pre-coated with poly-D-lysine, at a density of 7.5×10⁵ cells per well. After a 24 hour incubation, media was removed and replaced with a Ca²⁻ assay buffer (20 mM HEPES, 1× HBSS, 2.5 mM probenecid, and Ca²⁺ assay dye, pH 7.4) (FLIPR® Calcium Assay Kit; Molecular Device Corp, Sunnyvale, Calif.). After a 1-hour incubation at 37° C., during which the cells were allowed to take up the dye, fluorescence responses of cells were measured with a FLIPRTETRA (Molecular Device Corp; Sunnyvale, Calif.) device upon the addition of variable concentrations of tastant, or vehicle, in the presence of assay buffer (20 mM HEPES, pH 7.4, 1× Hanks Balanced Salt [Invitrogen; Carlsbad, Calif.] and 2.5 mM probenecid). After data acquisition, a subsequent addition of 5 mM thapsigargin was injected into each well, and fluorescence was measured again. Net peak responses to tastants were normalized to net peak responses to thapsigargin. Responses were compared with that of wild-type control MB9812, NCI-H520 or NCI-H522 cells. Statistical and graphical analyses were performed using Prism v. 5.0b (GraphPad Software; La Jolla, Calif.).

### Results

MB9812, NCI-H520 and NCI-H522 cells were selected for taste stimulation. The cells were loaded with fluorescent calcium-sensitive dye, treated with a variety of tastants, and monitored for intracellular calcium release with a FLIPR imaging device.

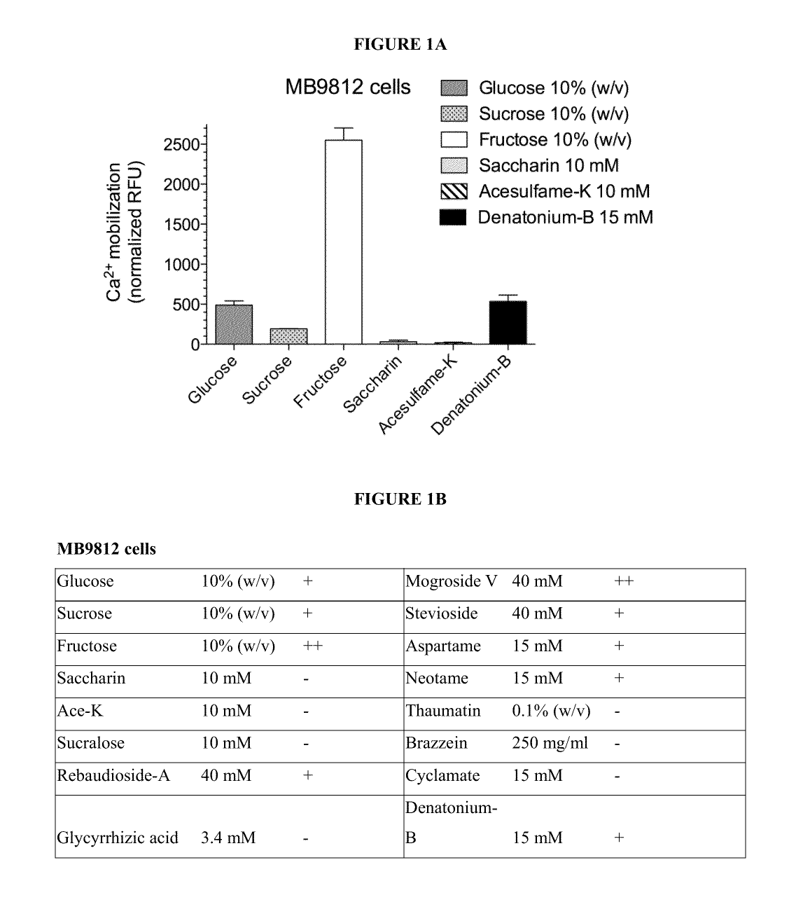

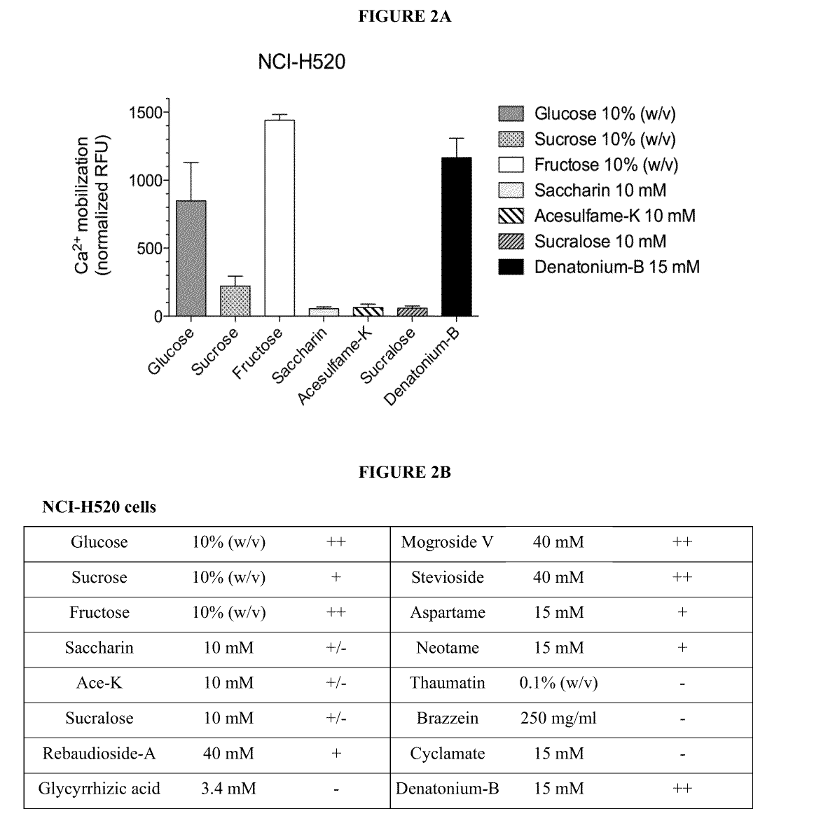

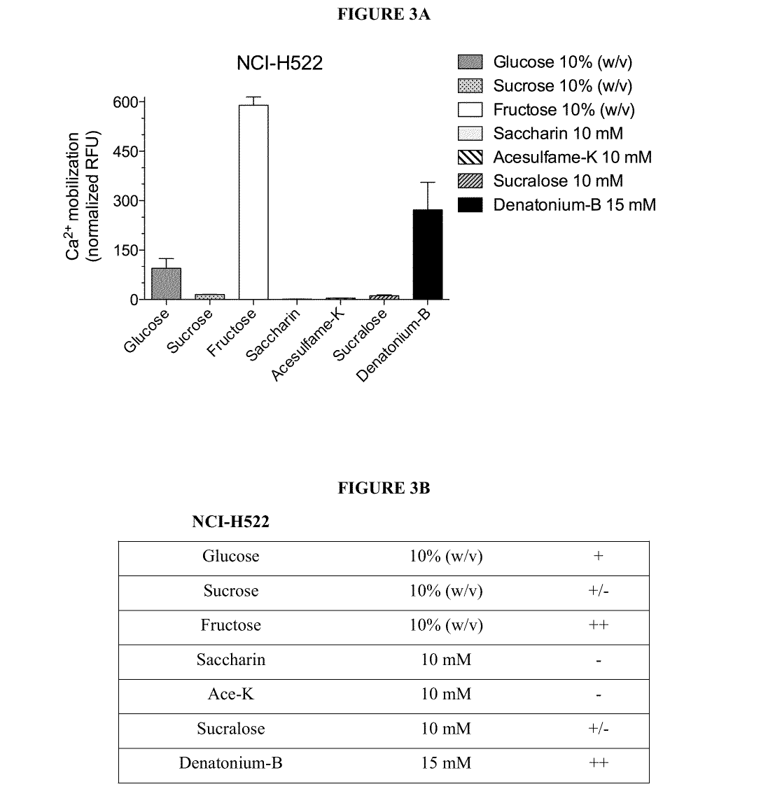

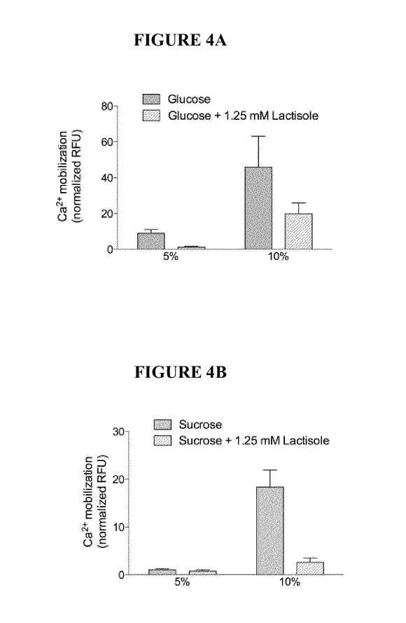

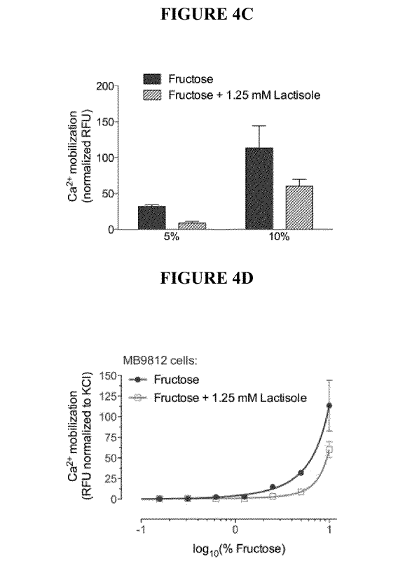

As shown in FIG. 1, MB9812 cells respond to the bitter compound, denatonium-B, and to sweeteners, as demonstrated by an increase in intracellular calcium. NCI-H520 cells and NCI-H522 cells also respond to denatonium B and sweeteners (see FIGS. 2A-B and 3A-B, respectively). FIGS. 4A-D show that sweetener response is effected via a lactisole sensitive receptor in MB9812 cells as evidenced by inhibition of the sweetener response by lactisole, a T1R3 inhibitor. Sweetener response was also inhibited by lactisole in NCI-H520 and NCI-H522 cells.

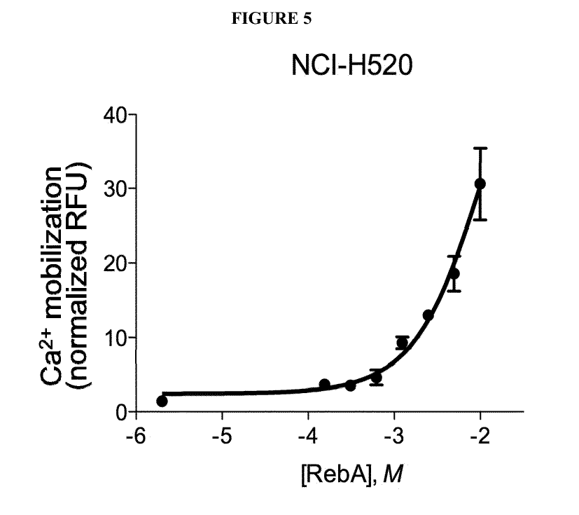

FIG. 5 shows that NCI-H520 cells respond to increasing concentrations of Rebaudioside A, the primary sweetener of Truvia. Similar results were obtained with MB9812 cells and NCI-H522 cells.

These results show that MB9812 cells, NCI-H520 cells and NCI-H522 cells can be used for the cell-based detection of bitterants, sweeteners, bitter taste modulators and sweet taste modulators.

What is claimed is:

1. A method for identifying a bitter taste modulator comprising:
a) contacting a cell with a bitter tastant and a test compound, wherein the cell is derived from airway tissue and endogenously expresses a bitter taste receptor and a sweet taste receptor;
b) measuring bitter taste receptor activity, wherein a change in bitter taste receptor activity by the bitter tastant indicates modulation of the bitter taste receptor by the test compound, thus identifying a bitter taste modulator.

2. The method of claim 1, wherein the cell endogenously expresses RGS21.

3. The method of claim 1, wherein the modulator inhibits the activity of the bitter tastant on the bitter taste receptor.

4. The method of claim 1, wherein the bitter taste receptor is T2R46 or T2R38.

5. The method of claim 1, wherein the cell is a MB9812 cell, a NCI-H520 cell, a NCI-H522 cell or derivative thereof

6. The method of claim 1, wherein the cell is modified to overexpress the bitter taste receptor.

7. The method of claim 1, wherein bitter taste receptor activity is measured by detecting the level of an intracellular second messenger in the cell.

8. The method of claim 7, wherein the second messenger is cAMP.

9. The method of claim 7, wherein the second messenger is DAG or IP3.

10. The method of claim 1, wherein bitter taste receptor activity is measured by detecting the level of intracellular calcium in the cell.

11. The method of claim 1, wherein the bitter taste receptor activity is binding activity.

12. The method of claim 11, wherein a change in binding activity is detected by a competitive binding assay.

13. The method of claim 11, wherein a change in binding activity is detected by surface plasmon resonance.

14. A method for identifying a bitter tastant comprising:
a) contacting a cell, wherein the cell is derived from airway tissue and endogenously expresses a bitter taste receptor and a sweet taste receptor with a test compound;
b) measuring bitter taste receptor activity, wherein an increase in bitter taste receptor activity indicates that the test compound is a bitter tastant.

15. The method of claim 14, wherein the cell endogenously expresses RGS21.

16. The method of claim 14, wherein the cell is a MB9812 cell, a NCI-H520 cell, a NCI-H522 cell or derivative thereof

17. The method of claim 14, wherein the bitter taste receptor is T2R46 or T2R38.

18. The method of claim 14, wherein bitter taste receptor activity is measured by detecting the level of an intracellular second messenger in the cell.

19. The method of claim 18, wherein the second messenger is cAMP.

20. The method of claim 18, wherein the second messenger is DAG or IP3.

21. The method of claim 14, wherein bitter taste receptor activity is measured by detecting the level of intracellular calcium in the cell.

22. The method of claim 14, wherein the bitter taste receptor activity is binding activity.

23. The method of claim 22, wherein binding activity is detected by a competitive binding assay.

24. The method of claim 22, wherein a change in binding activity is detected by surface plasmon resonance.

25. The method of claim 14, wherein the cell is modified to overexpress the bitter taste receptor.

26. A method for identifying a sweet taste modulator comprising:
a) contacting a cell with a sweetener and a test compound, wherein the cell is derived from airway tissue and endogenously expresses a bitter taste receptor and a sweet taste receptor;
b) measuring sweet taste receptor activity, wherein a change in sweet taste receptor activity by the sweetener indicates modulation of the sweet taste receptor by the test compound, thus identifying a sweet taste modulator.

27. The method of claim 26, wherein the cell endogenously expresses RGS21.

28. The method of claim 26, wherein the modulator inhibits the activity of the sweetener on the sweet taste receptor.

29. The method of claim 26, wherein the sweet taste receptor is T1R2/T1R3.

30. The method of claim 26, wherein the cell is a MB9812 cell, a NCI-H520 cell, a NCI-H522 cell or derivative thereof

31. The method of claim 26, wherein the cell is modified to overexpress the sweet taste receptor.

32. The method of claim 26, wherein sweet taste receptor activity is measured by detecting the level of an intracellular second messenger in the cell.

33. The method of claim 32, wherein the second messenger is cAMP.

34. The method of claim 33, wherein the second messenger is DAG or IP3.

35. The method of claim 26, wherein sweet taste receptor activity is measured by detecting the level of intracellular calcium in the cell.

36. The method of claim 26, wherein the sweet taste receptor activity is binding activity.

37. The method of claim 36, wherein a change in binding activity is detected by a competitive binding assay.

38. The method of claim 37, wherein a change in binding activity is detected by surface plasmon resonance.

39. A method for identifying a sweetener comprising:
a) contacting a cell, wherein the cell is derived from airway tissue and endogenously expresses a sweet taste receptor with a test compound;
b) measuring sweet taste receptor activity, wherein an increase in sweet taste receptor activity indicates that the test compound is a sweetener.

40. The method of claim 39, wherein the cell endogenously expresses RGS21.

41. The method of claim 39, wherein the sweet taste receptor is T1R2/T1R3.

42. The method of claim 39, wherein sweet taste receptor activity is measured by detecting the level of an intracellular second messenger in the cell.

43. The method of claim 42, wherein the second messenger is cAMP.

44. The method of claim 42, wherein the second messenger is DAG or 1P3.

45. The method of claim 39, wherein sweet taste receptor activity is measured by detecting the level of intracellular calcium in the cell.

46. The method of claim 39, wherein the sweet taste receptor activity is binding activity.

47. The method of claim 46, wherein binding activity is detected by a competitive binding assay.

48. The method of claim 46, wherein a change in binding activity is detected by surface plasmon resonance.

49. The method of claim 39, wherein the cell is modified to overexpress the sweet taste receptor.

50. The method of claim 39, wherein the cell further endogenously expresses a bitter taste receptor.

51. A method for identifying a sweet taste modulator comprising:
a) contacting a cell with a sweetener and a test compound, wherein the cell is derived from airway tissue and endogenously expresses a sweet taste receptor;
b) measuring sweet taste receptor activity, wherein a change in sweet taste receptor activity by the sweetener indicates modulation of the sweet taste receptor by the test compound, thus identifying a sweet taste modulator.

52. The method of claim 51, wherein the cell endogenously expresses RGS21.

53. The method of claim 51, wherein the modulator inhibits the activity of the sweetener on the sweet taste receptor.

54. The method of claim 51, wherein the sweet taste receptor is T1R2/T1R3.

55. The method of claim 51, wherein the cell is a MB9812 cell, a NCI-H520 cell, a NCI-H522 cell or derivative thereof

56. The method of claim 51, wherein the cell is modified to overexpress the sweet taste receptor.

57. The method of claim 51, wherein sweet taste receptor activity is measured by detecting the level of an intracellular second messenger in the cell.

58. The method of claim 57, wherein the second messenger is cAMP.

59. The method of claim 57, wherein the second messenger is DAG or IP3.

60. The method of claims 51, wherein sweet taste receptor activity is measured by detecting the level of intracellular calcium in the cell.

61. The method of claim 51, wherein the sweet taste receptor activity is binding activity.

62. The method of claim 61, wherein a change in binding activity is detected by a competitive binding assay.

63. The method of claim 61, wherein a change in binding activity is detected by surface plasmon resonance.

64. An isolated population of airway cells that express a bitter receptor and a sweet receptor, wherein the airway cells comprise an exogenous nucleic acid that encodes the bitter receptor and/or an exogenous nucleic acid that encodes the sweet receptor.

65. The population of claim 64, wherein the cells endogenously express RGS21.

66. An isolated population of airway cells that express a sweet receptor, wherein the airway cells comprise an exogenous nucleic acid that encodes the sweet receptor.

67. The population of claim 66, wherein the cells endogenously express RGS21.
